(19) United States
(12) Patent Application Publication
(10) Pub. No.: US 2011/0224690 A1
(43) Pub. Date: Sep. 15, 2011

(54) Tick Removal Instrument

(76) Inventors: Robert E. Henson, Piedmont, MO (US); John Henson, JR., Piedmont, MO (US)

(21) Appl. No.: 12/645,489

(22) Filed: Mar. 12, 2010

(51) Int. Cl. A61B 17/50

(52) U.S. Cl. 606/131

(57) ABSTRACT

It is of utmost importance that an imbedded tick is removed promptly after attachment and without causing distress to prevent possible infection from harmful diseases ticks carry. Further problems are created when the mouthparts are disassociated and left within the host's dermis. The Tick Removal Instrument is able to reliably remove the tick, including the mouthparts, without provoking the parasite to expel fluids into the host. The tick engaging portion includes a “vee groove” and a pointing prong which close upon each other to trap the tick. The pointing prong includes a specifically designed part which paralyzes the tick and causes it to release its grip from within the host's dermis. Ergonomic thumb and finger locators facilitate the use of the tool in a comfortable position suitable for use by either hand. The tick is easily removed by a gentle motion of the tool.

### CROSS-REFERENCE TO RELATED APPLICATIONS

### Not Applicable

### STATEMENT REGARDING FEDERALLY SPONSORED RESEARCH OR DEVELOPMENT

### Not Applicable

### BACKGROUND OF THE INVENTION

Field of Search

CLASS 128 SURGERY

CLASS 606 SURGERY (This Class, 606 is considered to be an integral part of Class 128)

### 1 INSTRUMENTS

131 Means for removal of skin or material there from

### 210 Tweezers

CLASS 294 HANDLING: HAND AND HOIST-LINE IMPLEMENTS 294-1

99.1 Resilient jaws

### 99.2 Hand-held

86.28 Outwardly biased grappling means

CROSS-REFERENCE ART COLLECTIONS

### 902 GRIPPING ELEMENT

897 MISCELLANEOUS

### 898 Methods

CLASS D24, MEDICAL AND LABORATORY EQUIPMENT

133 Hand manipulated tool or instrument (23)
143 Clamp or forceps (28)
147 Manually driven (32)

### 149 Opposed

Field of Search Classes

219/227, 219/233, 219/229, 294/99.2, 294/100, 606/206, 606/131, 606/133, 606/264, 606/27, 606/29, 606/42, 606/210, 606/211, 362/157, 362/204, 362/202, 362/208, 362/119, 43/134

1. Field of the Invention

The Tick Removal Instrument relates to devices for removing foreign objects from the surface of the skin; in particular, removing ticks from the skin of animals and humans alike. The present invention relates generally to the field of surgical instruments and devices for removing a parasite, namely a tick from a host's (human or animal) dermis.

2. Description of the Prior Art

In recent years, there has been an alarming increase in the incidence and geographical range of tick-born disease. Both humans and animals are affected by a number of infectious agents carried in and transmitted by ticks, such as Rocky Mountain Spotted Fever and Lyme Disease.

When a tick attaches to a human or an animal host, it not only inserts a portion of its mouthparts (chelicerae) into the host to reach the blood supply, it also secretes an adherent cement onto the skin of the host which helps prevent the tick from becoming detached from the host. After the tick attaches to the host, infectious agents within the tick may be transmitted to the host by the tick during its feeding cycle.

After attachment, as few as five (5) hours may elapse before infectious agents begin transmission; consequently, the sooner the tick is removed, the lower the risk of infectious agents being transmitted to the host. Therefore, the best method of protecting the host from any diseases carried by the tick is a rapid, effective, and complete removal of the tick from the host.

The recommended method for removal of a tick is to lift a tick out of the host with tweezers by securing the tweezers around the tick, as close to the host's skin as possible, and pulling with a steady, constant motion. The recommended method is not always effective in preventing disease transmission because the pulling motion can pull the tick apart, leaving the mouthparts and adhering cement in the host. The tick can be squeezed during attempted removal as well. This squeezing action forces gut fluids from within the tick into the host. These fluids may contain harmful or even disease-causing bacteria.

A more specific object of previous inventions is to provide a composition which aids in the removal of a tick from its host. Potentially, chemical agents may irritate the tick and thereby stimulate it to transmit infectious agents to the host.ⁱ The host may have an adverse reaction to the chemical agents. Another problem is that chemical agents that do stimulate the tick to detach from the host will not reliably remove the adherent cement excreted by the tick. It usually remains on the skin of the host after the tick detaches. The adherent cement may carry infectious agents itself or it may leave a wound in the skin of the host. The risk of infection is greatly increased.

Another major drawback of chemical agents is that they must be available for use within hours after the tick attaches. It is often not possible to bring a person or animal to a supply of the chemical agent quickly enough. Alternatively, it is unlikely that people involved in outdoor recreation will carry a supply of a tick removal agent with them at all times. They may be opposed to the usage of chemical agents. The usage of chemical agents on children or animals may sting, burn or otherwise be less than appealing to the host and cause the host to react violently against peaceful treatment.

Therefore, ideally, the best method to prevent transmission of disease from the tick to the host is complete removal of the tick and the adherent cement by mechanical means. A problem inherent in mechanical systems is that care must be exercised to avoid squeezing or crushing the body of the tick during removal because saliva or gut contents which may contain infectious agents can be regurgitated into the human or animal host.

In order to effect mechanical removal of the tick from the host, a number of prior art forceps and tweezers have been developed. One problem with forceps and tweezers of the prior art is that they rely on pressure against the body of the tick in order to obtain an adequate purchase for removal. In other words, they clamp the body of the tick like a pair of pliers. Any squeezing of the body of the tick may cause body fluids of the tick to be expelled into or onto the host. This increases the likelihood of exposure to dangerous elements from within the tick.

Another problem with the prior art tick removal forceps is that the tips are designed to clamp solidly together in continuous full surface contact. This makes removal of ticks in hair bearing areas or in the invitingly warm folds or creases of the host's skin difficult because the host's skin may be pinched or the host's hair may be pulled. Discomfort to the host makes removal of the tick more traumatic, particularly if the host is a child or an animal.

3. Background of the Prior Art

Ordinary tweezers and forceps are not suitable for properly removing embedded ticks from the dermis of humans or animals. Attempting removal of a tick by merely grasping the body and pulling (or twisting, as some prescribe for removal) nearly ensures leaving the mouthparts in the host's dermis. This increases the likelihood of infection by separating the body from the mouthparts of the tick, exposing the host to conditions such as Rocky Mountain Spotted Fever, Lyme Disease and other conditions yet to be adequately understood.

Ordinary tweezers and forceps have the distinct disadvantage of application of a squeezing force can be regulated only in a crude fashion by squeezing the apparatus closed with one's fingers. The squeezing force used can easily be too great forcing the tick to regurgitate its fluids into the victim's skin. If the head is too firmly attached to the skin, the result is generally decapitation of the parasite. Attempting to grasp the head while still embedded in the victim's skin poses further difficulties. It is common knowledge and expounded in the art that there is both risk and harm in severing or rendering the tick from its mouthparts during attempts to remove it.

The mouthparts are only a few tenths of a millimeter in diameter. The tick excretes a thick substance in which it effectively cements itself within the host's dermis. Ticks have a well defined division between the mouthparts and alloscutum. This makes the ease of separation even greater.

Some of the art is directed to professionals who can justify more expensive equipment. Many of the previous inventions are available to the casual user, (e.g. parents, pet owners, hunters or farmers) by using cheaper, simpler and more readily available materials. The present invention can be used by professionals and lay people alike. The previous art's lack of recognition of the ergonomic benefits and improved performance achievable through design innovations which combine the sensitivity available in natural bi-dexterous hand placement with complimentary structure which is a task-specific extension of the user's hand has impeded the natural progression of the invention process.

### BRIEF SUMMARY OF THE INVENTION

The main purpose of the tool is to provide a simple means for the intact removal of a tick or similar parasite which has its head and jaws embedded in the skin of a host animal or person. The Tick Removal Instrument is a bi-dexterous tool which can be readily grasped and manipulated by either hand. To this end, locators are provided on opposing sides of the handle suitable for thumb and forefinger positioning and actuation.

The invention provides a hand tool that employs a natural, palm-down, hand position and gripping motion closely simulating the motions that might be used to do the job with one's own fingers. One is able to manipulate the tool with one hand without excessive arm or wrist motion, while maintaining a substantially unobstructed view of the tick.

Further, there is a minimal parts count. The Tick Removal Instrument may be molded as a single piece tool. The present invention can be made out of a number of materials such as stainless steel, metal alloy or plastic.

The Tick Removal Instrument's precisely designed head and exacting location of the “Paralyzer” safely removes the chelicerae or mouthparts from within the host. This is to prevent contamination of the host by the fluids contained within the body of the tick. When the Tick Removal Instrument is used properly, according to instructions, the head is removed every time. There is no invention to this date that can adequately perform to this standard. One can see the remaining cementing fluids attached to the tick's chelicerae when the tick is excised from within the victim's skin.

The Tick Removal Instrument is configured and sized to substantially enclose the body of the parasite when the invention is in the closed position. The apparatus is sized to closely surround the mouthparts of the parasite when the arms are in the closed position. The Tick Removal Instrument is designed to ensure the tick is left intact for proper disposal or investigation.

The active portions of the invention are mounted on the opposite end of the opposing arms. The 4-¼″ conjoined handles provide for precision extraction every time the Tick Removal Instrument is used according to directions provided.

### BRIEF DESCRIPTION OF THE SEVERAL VIEWS OF THE DRAWING

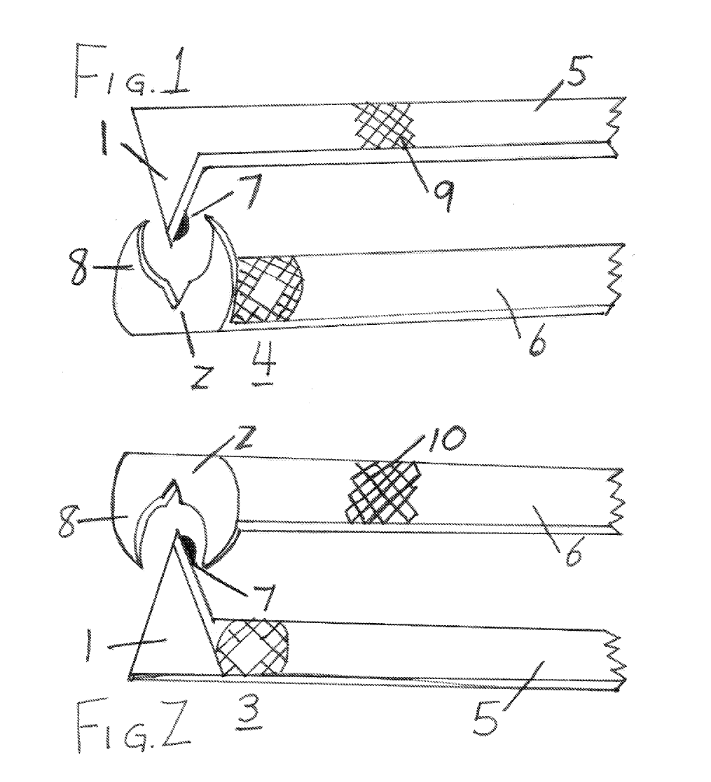

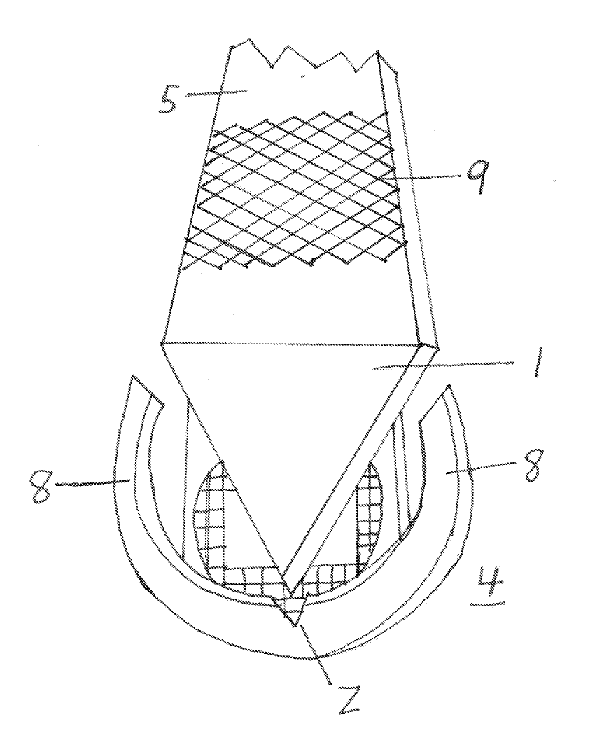

Drawing 1/4, FIG. 1 Side View Open position

A side elevation of the Tick Removal Instrument legs Part 1 and Part 2 in an open position

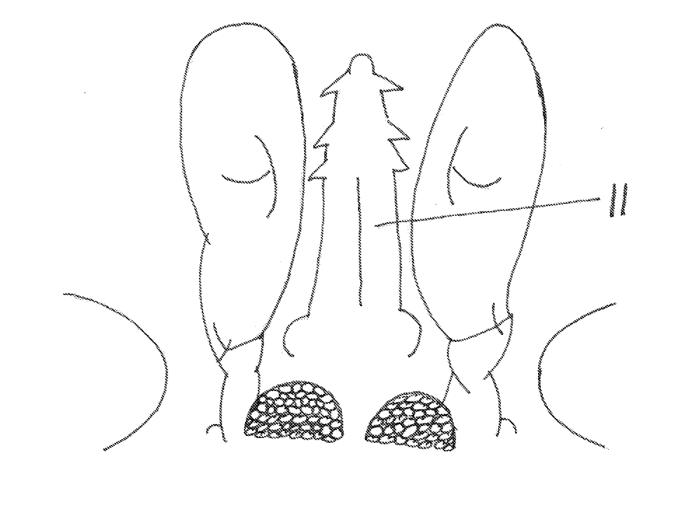

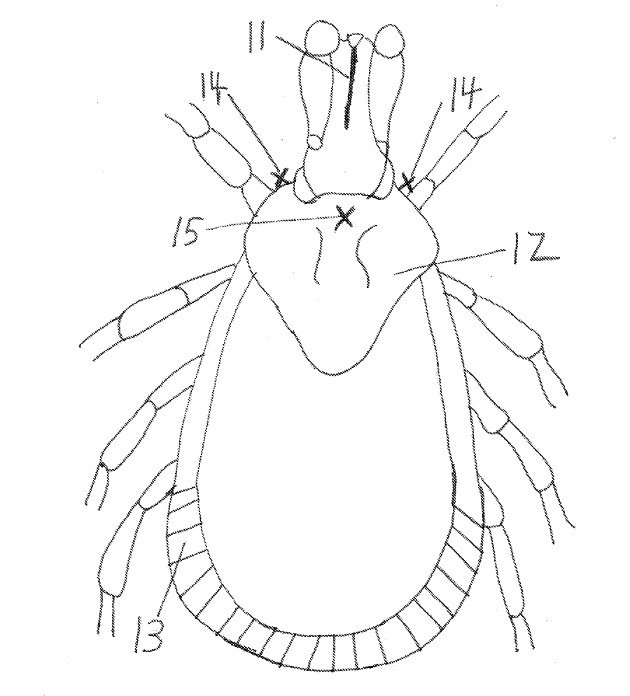

Drawing 1/4, FIG. 2 Side View Open Position

A side elevation of the invention included for the express purpose of portraying visual angles not available in FIG. 1.

Drawing 2/4, End View Closed position

Final closing of the gripping points takes place

Drawing 3/4, Sketch of Scanning Electron Microscope close-up of tick's chelicerae or mouthparts

Illustration showing in magnification mouthparts of common tick

Drawing 4/4, Sketch of Representative Tick

Sketch of a basic tick. Example of points of contact where the final closing of the Tick Removal Instrument's gripping points takes place

### DETAILED DESCRIPTION OF THE INVENTION

Drawings 1, FIGS. 1 & 2, FIG. 1 are constructed according to the present invention. It relates to the manual removal from the host of live ticks whose mouthparts are embedded within the host. The head of the tick is smaller than the body. The body of the tick may or may not be engorged depending upon the stage of the feeding cycle that the tick is in.

We intend to protect our invention, The Tick Removal Instrument, by patenting it. The Tick Removal Instrument is the best product available for the safe removal of ticks. It is the only tool which reliably removes the chelicerae or mouthparts from within the host's skin. By not squeezing the tick during the removal process using this instrument, regurgitation of body fluids that may contain harmful bacteria or diseases are mitigated. The Tick Removal Instrument is the only invention that can say this.

The Tick Removal Instrument is intended to be made of plastic, stainless steel or a metal alloy. It will be injection-molded in one piece. The thickness chosen for the Tick Removal Instrument permits controlled bending and flexing throughout the conjoined handle section as desired by the user.

A stainless steel or metal alloy model would be useful when sterilization prior to each usage is required as in a medical office or in usage by an outdoors person, farmer or pet owner. The plastic form of Tick Removal Instrument would be useful in a medical or veterinary office where one-time usage would be the best course of action.

Tick Removal Instrument has an upper arm, Part 1 and a lower arm, Part 2 consisting of two equal length arms attached by a conjoined handle. Instrument parts are as follows.

Part 1 Upper Arm {1} The upper arm or Part 1 of the Tick Removal Instrument has a downward, yet slightly inward pointing prong (ref #1) extending at an angle of 15° from the handle of the Tick Removal Instrument (ref #5). Part 1 (ref #1) is 25/1000″ wide at the most extensive part and has a 15/1000″ width near the tip. The tip is machined such as to prevent it from completely entering the “vee groove” and severing the mouthparts from the rest of the body. There is a gap of 6/1000″+/− 1/1000″ inside the “vee groove” between the “vee groove” and the prong (ref #1) when the Tick Removal Instrument is in a full closed position.

{3} The central location of Part 1 (ref #3) is machined 15/1000″ wide and 6/1000″ deep to form a cavity which clutches the tick's body during removal. This concavity (ref #3) of central Part 1 is located immediately behind the distal end of the instrument containing the prong tip (ref #1) and just prior to the handle (ref #5) of the Tick Removal Instrument. The Instrument's width is 25/1000″ at the location of the cavity (ref #3).

{5} The single prong of Part 1 (ref #1) is located distally to the handle of Part 1 (ref #5) of the Tick Removal Instrument. The Tick Removal Instrument's handle is to be horizontally flat at a 90° angle and maintains a width of 25/1000″ throughout. The handle (ref #5) is intended to be conjoined to Part 2 handle (ref #6) of the Tick Removal Instrument in a tweezers-like manner.

{7} The “Paralyzer” begins 20/1000″ back from the tip of Part 1 and extends to a point 1/20″ from tip and is 18/1000″ thick +/− 2/1000″. The width of the “Paralyzer” is the same as the width of the tip of Part 1 (ref #1), 15/1000″. The “Paralyzer” is located on the underneath side of Part 1 tip (ref #1). It is a calculated placement of the same material the Tick Removal Instrument is made of. The purpose of the “Paralyzer” is to engage the back of the tick's alloscutum during removal from the host.

Upon application of pressure provided by the actuation of Tick Removal Instrument, the tick expediently releases its grip from within the host's dermis. The application of pressure is theorized to be paralytic to the tick. At the time of our research for this patent, there was no other device which is capable of complete coercion of the tick in removal its hold on the host. This paralyzation occurs immediately upon actuation of the Tick Removal Instrument. It is evident due to the tick's immediate straightening of its front legs out and away from the body. The cement-like substance secreted by the tick is quickly liquefied by the tick. This aids in the complete, safe removal of the mouthparts from within the host's dermis

{9} An ergonomic thumb and finger locator is on the outer surface of the upper arm, Part 1 of the Tick Removal Instrument. Cross hatches machined into the surface of the instrument provide a placement guide and firm gripping surface for the user.

Part 2 Lower Arm {2} Part 2 or the bottom arm of the Tick Removal Instrument consists of two (2) upward, yet slightly outward pointing prongs (ref #2) extending at an angle of 15° from Part 2 arm (ref #6). The arm is 25/1000″ wide at the most extensive part. A “vee groove” is fashioned into the instrument's Part 2 arm such as to be narrower than the tip of Part 1. This prevents the tip of Part 1 from entering the “vee groove” of Part 2 and severing the head from the rest of the body. The purpose of the “vee groove” is to secure the mouthparts during the removal of the feeding tick from the host's dermis.

{4} A cavity (ref #4) is formed within central Part 2 and is machined to a width of 15/1000″ and 6/1000″ deep to firmly grasp the tick's body during removal by the Tick Removal Instrument. This Concavity (ref #4) is located immediately behind the distal end of the instrument containing the dual prong end (ref #2) and just before the handle (ref #6) of the Tick Removal Instrument.

{6} The dual prongs of Part 2 (ref #2) are distally located to handle of Part 2 (ref #6) of the Tick Removal Instrument. The handle is straight and lies at a 90° horizontal angle and maintains a width of 25/1000″ throughout. The handle is intended to be joined in a tweezers-like manner to the handle of Part 1 (ref #5).

{8} The “ears” of the “vee groove” located on Part 2 of the Tick Removal Instrument engages the tick on either side of its mouthparts and actually touches the tick's alloscutum. The concavity of (ref #4) holds the tick steady while the “ears” of the “vee groove” provide leverage for pulling the tick out and away from the host at its feeding insertion site.

{10} Ergonomic thumb and finger locator is on the outer surface of the lower arm, Part 2 of the Tick Removal Instrument. Cross hatches machined into the surface of the instrument provide a placement guide and firm gripping surface for the user.

Height of closed tick removal instrument is ⅛″, Part 1 and Part 2 Width of closed tick removal instrument 3/32″, Part 1 and Part 2 The thickness chosen for the Tick Removal Instrument preferably permits controlled bending and flexing throughout the handle section as desired by the user. The thickness of the distal end of Part 1 and Part 2 of the tick Removal Instrument (ref #1&2) is 12/1000″+/− 2/1000″. The thickness of instrument near the holding cavities (ref #3 & 4) are 18/1000″+/− 2/1000″. The Tick Removal Instrument has a 24/1000″+/− 2/1000″ thickness at the handle ends of Part 1 and Part 2 (ref #5 & 6).

The tick as it is described and sketched herein contains generalities as to body sections as currently accepted by professionals in the field.

{11} Indicate the chelicerae or mouthparts of a basic tick. The chelicerae cut into the dermis providing an opening for the serrated hypostome to enter the wound. The chelicerae hold the tick's position within the opening it has formed. The tick releases an agent to block coagulation. The tick then secretes a sticky substance which effectively cements the mouthparts in place. When the mouthparts become detached from the rest of the body, they are especially problematic to remove. Generally, the only recourse is to wait for the host's body to react to the foreign item, swell and begin dispensing the mouthparts.

{12} Alloscutum of a basic tick. One might think of this portion of a tick as a thorax. The anterior of the alloscutum is where the “ears” of the Tick Removal Instrument gently pull backwards (ref #14) during removal. The ventral side of the tick lies securely within the concavity of Part 1 (ref #3) of the Tick Removal Instrument. The anterior dorsal central area of the scutum is where the “Paralyzer” (ref #7) strikes the back of the tick (ref #15) forcing the tick to release its hold of the host.

{13} Festoons of a basic tick. The rest of the body from the alloscutum towards the rear is referred to as the Festoons.

{14} These are the Points of Contact of the Tick Removal Instrument on the front of a basic tick's alloscutum. These points are where the “ears” (ref #8) of the lower arm, Part 2 of the Tick Removal Instrument touch the tick during removal. It works to pull the tick gently away from the victim's dermis.

{15} Point of Contact for the “Paralyzer” (ref # 7) of Part 1 of upper arm of the Tick Removal Instrument. Pressure applied from the Tick Removal Instrument causes the tick to release its grip from within the victim's dermis; the cement-like substance is fluidized and the tick is removed completely.

1. The Tick Removal Instrument is a tool created for the express purpose of removing ticks completely, to include their chelicerae, from their host's dermis without promoting the transmission of harmful fluids from within the tick into the victim.
By not squeezing the tick during the removal process, the regurgitation of body fluids which may contain harmful bacteria or diseases are mitigated.

2. The closure of the Tick Removal Instrument effectively paralyzes the tick by application of the “Paralyzer” upon the back of the tick's alloscutum.
This paralyzing effect is evident by the sudden straightening of the tick's front legs.
The pinning action of the actuation of the Tick Removal Instrument forces the tick to release its grip.
The pinning action simultaneously causes the tick to dissolve the cementing substance which bonds the tick's mouthparts within the host's dermis.
